(19) United States
(12) Patent Application Publication
(10) Pub. No.: US 2017/0225405 A1
(43) Pub. Date: Aug. 10, 2017

(54) IN-SITU FABRICATION OF A POROUS SCAFFOLD

(71) Applicants: Nasim Safdarian, Tehran (IR); Hamid Mobedi, Tehran (IR); Franz E. Weber, Zurich (CH)

(72) Inventors: Nasim Safdarian, Tehran (IR); Hamid Mobedi, Tehran (IR); Franz E. Weber, Zurich (CH)

(21) Appl. No.: 15/498,458

(22) Filed: Apr. 26, 2017

Related U.S. Application Data
(60) Provisional application No. 62/346,560, filed on Jun. 7, 2016.

(51) Int. Cl. B29C 67/20; A61F 2/02; A61L 27/54; A61L 27/18; A61L 27/58; A61F 2/00; A61L 27/56

(52) U.S. Cl. CPC B29C 67/202; A61F 2/0077; A61F 2/02; A61L 27/56; A61L 27/18; A61L 27/58; A61L 27/54; B29K 2995/0056; B29K 2995/006; B29L 2031/753

(57) ABSTRACT

A method includes mixing a polymer, an organic solvent, and a porogen such that an initial paste is formed. The method also includes in-situ shaping the initial paste; creating a plurality of channels within the shaped paste and removing the organic solvent from the shaped paste such that a solidified perforated paste is formed; and leaching out the porogen from the solidified perforated paste such that a porous scaffold is formed.

### CROSS REFERENCE TO RELATED APPLICATION

This application claims the benefit of priority from pending U.S. Provisional Patent Application No. 62/346,560, filed on Jun. 7, 2016, and entitled “A METHOD FOR CREATING UNIFORM POROSITY IN IN-SITU FORMING SCAFFOLDS,” which is incorporated herein by reference in its entirety.

### TECHNICAL FIELD

The present disclosure generally relates to tissue engineering, particularly to porous scaffolds, and more particularly to a method for in-situ fabrication of porous scaffolds.

### BACKGROUND

In tissue engineering, a scaffold is used to provide a proper substrate for cell attachment, cell proliferation, cell differentiation, and cell migration. To obtain a proper tissue ingrowth and ensuring of nutrient and cell delivery using an implanted scaffold, the scaffold must meet certain specifications.

Since the scaffold fabrication methods should allow for the control of its pore size and pore interconnectivity, and should enhance the maintenance of its mechanical properties, several methods have been developed to fabricate porous scaffolds for tissue engineering, including fiber bonding, solvent casting and particulate leaching, particle sintering, three-dimensional printing, gas foaming, emulsion freeze drying, and phase separation.

Hence, there is a need for a fabrication method allowing the control of the morphology, void volume and pore size distribution of the void volume and to achieve a uniform pore distribution with an appropriate interconnectivity in porous scaffolds.

### SUMMARY

In one general aspect, the present disclosure describes a method for in-situ fabricating a porous scaffold. The method may include the steps of: mixing a polymer, an organic solvent and a porogen to form an initial paste, in-situ shaping the paste to form a shaped paste, creating a plurality of channels within the shaped paste and removing the organic solvent from the shaped paste to form a solidified perforated paste, and leaching out the porogen from the solidified perforated paste to form the porous scaffold. In certain implementations, the steps of creating the plurality of channels within the shaped paste and removing the organic solvent may be performed substantially simultaneously. In other implementations, the step of removing the organic solvent may be performed after the step of creating the plurality of channels. In some cases, the step of creating the plurality of channels may be repeated while the organic solvent is being removed. In certain examples, the step of creating the plurality of channels may be performed during solidification of the shaped paste

The above general aspect may include one or more of the following features. In some examples, the porous scaffold may have a pore size between about 50 micrometers and about 1000 micrometers. The porous scaffold may have a uniform porosity and/or high pore interconnectivity.

According to some implementations, the polymer may be a biodegradable and biocompatible polymer and the polymer may be for example polylactide (PLA), poly (lactide-co-glycolide) (PLGA), polycaprolactone (PLC), polyglycolide (PGA), polyanhydrides, polydioxanone, polyorthoester, polyurethane, polyacrylate, polycarbonate, polysiloxane, polyolefin, polyamide, polyimide, poly alpha hydroxyl acids, or combinations thereof. In certain examples, the polymer may have a molecular weight between about 2000 Daltons and about 50000 Daltons.

According to some implementations, the porogen may include sodium chloride, potassium chloride, potassium bromide, calcium chloride, magnesium chloride, polyethylene glycol, polypropylene glycol, sorbitol, sucrose, poly-carbohydrate, gelatin, sugar, mannitol, poly vinyl alcohol, or combinations thereof. Also, in some cases, the porogen may be soluble in water and insoluble in the organic solvent. In some implementations, the porogen may have a particle size between about 100 micrometers and about 1500 micrometers. Moreover, the concentration ratio (weight/weight) of the polymer to the porogen may be between for example 1:1 (wt/wt) and 1:10 (wt/wt).

According to some implementations, the organic solvent may be a water-miscible solvent and the organic solvent may be for example acetone, chloroform, dimethyl carbonate, N-Methyl-2-pyrrolidone, dimethyl sulfoxide, polyethylene glycol, polyethylene glycol derivatives, or mixtures thereof. The concentration ratio (weight/weight) of the polymer to the organic solvent may be between for example 1:1 and 1:10.

According to some implementations, creating the plurality of channels within the shaped paste is performed using one or more needles, and in certain cases, one of the needles may have a needle gauge between 10 and 34. In certain cases, removing the organic solvent from the shaped paste includes a phase inversion process.

According to an implementation, the method for in-situ fabricating a porous scaffold may further include adding an active agent to the initial paste. In some examples, the active agent may be an agent for cell induction or cell promotion. In certain cases, the active agent may include growth factors, anti-inflammatory agents, inductive agents, angiogenesis factors or tissue growth promoting agent

### BRIEF DESCRIPTION OF THE DRAWINGS

FIG. 1 illustrates an example of a method for in-situ fabrication of a porous scaffold, pursuant to one or more teachings of the present disclosure.

FIG. 2 illustrates an optical microscopic image of a cross section of an example of the in-situ fabricated porous scaffold with a uniform porosity, consistent with one or more implementations of the present disclosure.

FIG. 3 illustrates an optical microscopic image of a cross section of a scaffold with a non-uniform porosity, fabricated by using an example method not including a step of creating channels during the solidification of the paste.

FIG. 4A illustrates an optical microscopic image of an example of an in-situ fabricated porous scaffold with a magnification of 47.

FIG. 4B illustrates an optical microscopic image of an example of an in-situ fabricated porous scaffold with a magnification of 200.

FIG. 5 illustrates an optical microscopic image of an example of an in-situ fabricated porous scaffold after an interconnectivity assay.

FIG. 6 illustrates a diagram of compressive strength and compressive modulus of sample in-situ fabricated porous scaffolds at time points of 24, 48, and 72 hours after the fabrication.

### DETAILED DESCRIPTION

The following detailed description is presented to enable a person skilled in the art to make and use the methods and systems disclosed in various implementations of the present disclosure. For purposes of explanation, specific nomenclature is set forth to provide a thorough understanding of the present disclosure. However, it will be apparent to one skilled in the art that these specific details are not required to practice the disclosed sample embodiments. Descriptions of specific embodiments are provided only as representative examples. Various modifications to the disclosed implementations will be readily apparent to one skilled in the art, and the general principles defined herein may be applied to other implementations and applications without departing from the scope of the present disclosure. The present disclosure is not intended to be limited to the implementations shown, but is to be accorded the widest possible scope consistent with the principles and features disclosed herein.

Appropriate porosity is an important design criterion for scaffolds used in tissue engineering applications as it can permit increased cell adhesion, migration, proliferation and extracellular matrix production within the scaffold at a tissue defect site; therefore fabricating a porous scaffold with proper characteristics is crucial in the case of tissue engineering.

With the rationale of obviating some the limitations of current scaffold fabrication approaches, such as non-uniform porosity and poor interconnectivity, disclosed herein is a method for fabricating porous scaffolds with a uniform porosity and high interconnectivity which used in tissue engineering.

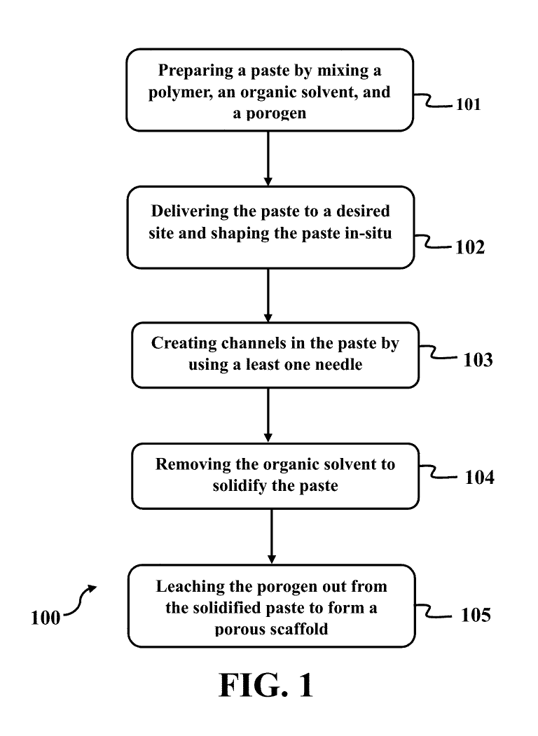

FIG. 1 is a flowchart of an example of a method for in-situ fabricating a porous scaffold, consistent with sample embodiments of the present disclosure. According to this figure, method 100 may include: mixing a polymer, an organic solvent and a porogen to form an initial paste (step 101), in-situ shaping the initial paste to form a shaped paste (step 102), creating a plurality of channels within the shaped paste (step 103) and removing the organic solvent from the shaped paste (step 104) to form a solidified perforated paste, and leaching out the porogen from the solidified perforated paste to form the porous scaffold (step 105). In certain implementations, steps 103 and 104 including creating a plurality of channels within the shaped paste and removing the organic solvent may be performed substantially simultaneously. In other implementations, step 104 of removing the organic solvent may be performed after step 103 of creating the plurality of channels. In some of these implementations, step 103 of creating the plurality of channels may be repeated while the organic solvent is being removed. In some cases, step 103 of creating the plurality of channels may be performed during solidification of the shaped paste.

Referring to step 101, a polymer, an organic solvent and a porogen may be mixed together to form a paste. In an implementation, for preparing the paste, a polymer may be mixed with an organic solvent to form a viscous mixture. Then, a porogen may be added to the viscous mixture to obtain the paste.

As used herein, the term of “porogen” refers to any of a mass of particles, of a specified shape and size, used to make pores in structures in tissue engineering applications.

In an implementation, the polymer may be a biodegradable and biocompatible polymer, for example, polylactide (PLA), poly(lactide-co-glycolide) (PLGA), polycaprolactone (PLC), polyglycolide (PGA), polyanhydrides, polydioxanone, polyorthoester, polyurethane, polyacrylate, polycarbonate, polysiloxane, polyolefin, polyamide, polyimide, poly alpha hydroxyl acids, and combination thereof. In certain examples, the polymer may have a molecular weight between about 2000 Daltons and about 50000 Daltons.

As used herein, the term of “biodegradable and biocompatible polymer” refers to a polymer which is not harmful or toxic to living tissues or biological systems.

According to some implementations, the organic solvent may be a water-miscible solvent such as, for example, acetone, chloroform, dimethyl carbonate, dimethyl sulfoxide, polyethylene glycol, polyethylene glycol derivatives, N-Methyl-2-pyrrolidone (NMP), dimethyl sulfoxide, polyethylene glycol, polyethylene glycol derivatives, or mixtures thereof.

In some implementations, a specific amount of a porogen may be sieved and the porogen particles with a particle size between for example, about 100 micrometers and about 1500 micrometers may be separated to be mixed with the polymer and the organic solvent in step 101. Then, the sieved porogens may be added to the mixture of the polymer and the organic solvent to form a homogenous paste.

In one implementation, the porogens may have a regular or irregular shape and may be for example, sodium chloride, potassium chloride, potassium bromide, calcium chloride, magnesium chloride, polyethylene glycol, polypropylene glycol, sorbitol, sucrose, poly-carbohydrate, gelatin, sugar, mannitol, poly vinyl alcohol (PVA), or their combinations. In certain cases, the porogen may be soluble in water and it may be insoluble in the organic solvent. The porogens may have a particle size between for example, about 100 micrometers and about 1500 micrometers.

In some implementations, the polymer may be mixed with the organic solvent in a concentration ratio (weight/weight) between for example, about 1:1 (wt/wt) and about 1:10 (wt/wt). Moreover, in some cases, the concentration ratio (weight/weight) of the polymer to the porogen may be between for example, about 1:1 (wt/wt) and about 1:10 (wt/wt).

In another implementation, the method 100 may further include adding at least one active agent to the paste. The active agent may be an agent for cell induction or cell promotion and may be for example a growth factor, an anti-inflammatory agent, an inductive agent, an angiogenesis factor, or a tissue growth promoting agent.

In step 102, the paste may be shaped in-situ to form a shaped paste. Accordingly, the paste prepared in step 101 may be introduced to a site, for example a mold or a biological site of a biological system. In certain examples, the biological site may include defects in organisms, or surfaces of a portion of musculoskeletal system, for example, a portion of a bone. After that, the paste may be shaped properly by using hand or any objects like spatula.

In step 103, a plurality of channels may be created within the shaped paste to form a perforated paste. In certain examples, channels may be created by using at least one needle and the needle may be a narrow rod object or a needle-like object. The needle may be for example, a syringe needle with a gauge between about 10 and about 34. In some cases, the paste may be perforated or punched in different directions, and the number of channels may be between for example 3 to 10 channels per square centimeter. In some examples, the channels may be open-ended channels.

As used herein, the term of “gauge” refers to the thickness of the needle as a standard measure.

In step 104, the organic solvent may be removed from the shaped paste to form a solidified perforated paste. For example, solidification of the paste may occur in a phase inversion process, which is a liquid-to-solid phase transition, by providing an aqueous environment for the perforated paste. The aqueous environment may be provide by adding an aqueous solution. The organic solvent, which in some examples is a water-miscible solvent, may dissipate out of the perforated paste and the water may diffuse into the paste; therefore, the solidification of paste may be obtained in a few minutes after the addition of aqueous solution and this exchange of solvents may lead to the scaffold formation.

According to an implementation, the aqueous solution may be for example, pure water, a buffer solution, a physiologic serum, etc. In certain cases, the amount of the aqueous solution may be from about 5 to about 100 ml, and the addition of the aqueous solution may be added to the paste by using a syringe during solidifying in step 104 in certain examples.

Referring again to method 100 illustrated FIG. 1, the steps 103 and 104 may be repeated until the whole paste becomes solid and a solidified perforated paste is obtained. Accordingly, creating the channels in the paste and simultaneous removal of the organic solvent may be repeated for about 3 to about 10 times, thereby solidifying the paste and forming the scaffold in-situ.

In 105, the porogen may be leached out from the solidified perforated paste to form the porous scaffold. The porogen may be leached out, for example, using a porogen-leaching process which involves the removal of the porogen from the solidified perforated paste for generating porosity. Therefore, the water-soluble porogens may be easily discharged into an aqueous solution through the channels and cause to form a porous scaffold. Dissolving the porogens in an aqueous solution may take some hours to some days and after that, a network with appropriately high porosity may be fabricated in-situ. The obtained porous scaffold may be applicable for the penetration of biological cells and other materials such as active agents or nutrients into the porous scaffold. In some examples, the porous scaffold may have a pore size between about 50 micrometers and about 1000 micrometers.

Without being bound by any theory, the present inventors believe that upon adding water on to the paste to start the phase inversion, the polymer starts solidifying from the surface of the paste. Inconsistency in the rate of phase inversion in the surface and depth of the scaffold may hamper the exit of the porogen from the depth of the scaffold, and consequently pores with a non-uniform distribution may appear across the scaffold. In the view of this, the present inventors believe that creating channels in the paste during its solidification, provides the possibility of better phase inversion in the whole scaffold and may prevent the lack of porosity in the central parts of the scaffold, since the channels help the porogens to be leached out. As a result, a scaffold with a uniform porosity and a high pore interconnectivity may be obtained.

### EXAMPLES

### Example 1: In-Situ Fabrication of a Porous Scaffold

In this example, a porous scaffold was fabricated in-situ assisted by a method according to one or more aspects of the present disclosure as described in details hereinabove.

At first, poly (lactide-co-glycolide) (PLGA) as a biodegradable and biocompatible polymer was mixed with N-Methyl-2-pyrrolidone (NMP) as an organic, water-miscible solvent by stirring overnight to form a viscous mixture. Thus, the concentration ratio (weight/weight) of the PLGA to NMP was about 1:5.

After that, sugar as a porogen was sieved and the sugar particles having a particle size between about 550 μm and about 750 μm were selected. Then, the selected sugar particles were added to the viscous mixture of the polymer and the organic solvent to form a homogenous paste. The concentration ratio (weight/weight) of the PLGA to the sugar was about 1:6.

In the next step, for delivering the paste to a desired site like the surface of a bone that has a defect, and shaping the paste in-situ, the prepared paste was introduced to a defect and then, the paste was shaped properly. After that, a plurality of channels, in a ratio of about 5 channels per square centimeter of the external surface area of the paste, were created in different directions throughout the entire paste using one syringe needle with a gauge of about 20 while about 20 ml of physiologic serum was added to the paste in order to solidify the paste. After two minutes exposure of the paste to the aqueous environment of physiologic serum, NMP was dissipated out of the paste in a constant rate across the surface into the depth of the paste and the physiologic serum diffused into the paste and this exchange of solvents, which was not limited just to the surface, led to the scaffold formation.

In order to create appropriate channels and to prevent rapid solidification of the paste, creating a plurality of channels in the paste and the addition of physiologic serum described above were done substantially simultaneously and were performed four times until the paste was completely solidified. About 80 ml of the physiologic serum was used in total.

Finally, in order to make an efficient porosity, the porogens which were soluble in the water, were leached out from the solidified perforated paste through a porogen-leaching process. Accordingly, pure water was added to the solidified perforated paste and then the porogens were dissolved in the aqueous environment throughout the channels in two days. Finally, a suitable network with appropriate porosity for the penetration of cells and other materials into the scaffold was fabricated.

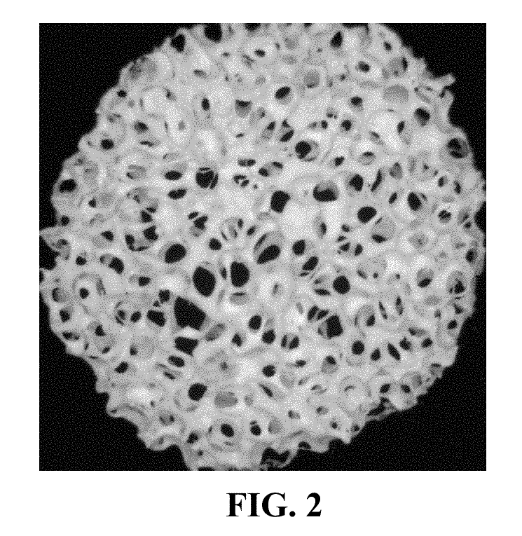

FIG. 2 shows the cross section of an example of the in-situ fabricated porous scaffold by using the method described in this example which involves creating a plurality of channels during the solidification of the paste. Referring to FIG. 2, the morphology of the scaffold is uniform throughout, contributing to the formation of spherical pores with definite and uniform dimensions. The scaffold has a high porosity and high pore interconnectivity which provide a good condition for the entry and penetration of cells, and growth thereof.

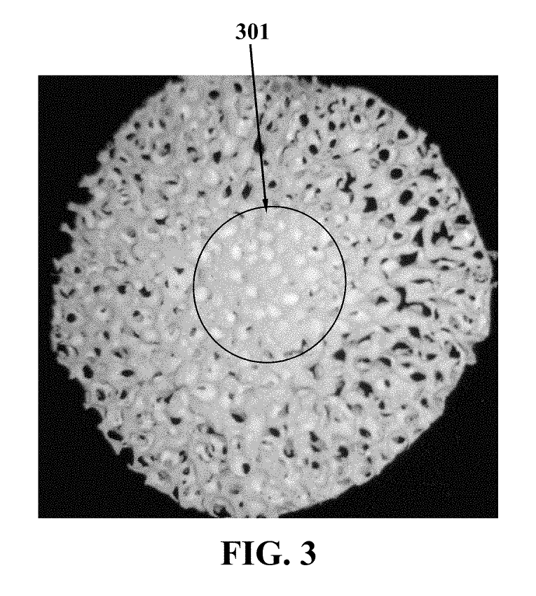

For the purpose of comparison, FIG. 3 shows the cross section of a scaffold with a non-uniform porosity which is fabricated by using a similar method, but, with lack of the step of creating channels during the solidification of the paste. Referring to FIG. 3, apparent difference in the distribution of porosities between the central part 301 of the scaffold and its surrounding part may be attributed to a non-homogenous phase inversion and leaching out the porogens from the scaffold.

### Example 2: Structural and Mechanical Characterization of the Porous Scaffold

In this example, porosity, pore interconnectivity, and mechanical analysis of the porous scaffold were assessed. Evaluating the porosity of the scaffold was done by using a simple gravimetric analysis which compares the density of porous scaffold with the density of non-porous polymer and calculate the porosity with a formula including:

po=1−d/dp, where po is porosity, d is density of the porous scaffold and dp is density of non-porous polymer.

According to the results of the gravimetric analysis, the mean average of total porosity of example scaffolds, pursuant to the teachings of the present disclosure, was about 86.63%.

The range of the pore size and the mean pore size were corresponding to the porogen size which was used in the method. In addition, the pore size of the scaffold was measured by using a DinoXcope software. The range of the pore size was reported from about 570 micrometers to about 760 micrometers; and the mean pore size of the scaffold was about 691 micrometers; therefore, on this scale, the PLGA in-situ fabricated scaffold possessed a uniform pore size distribution.

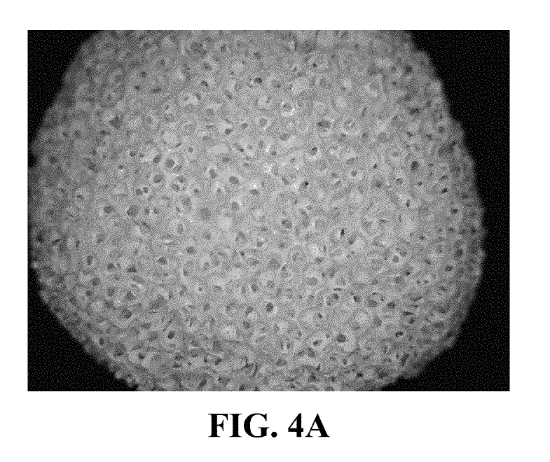

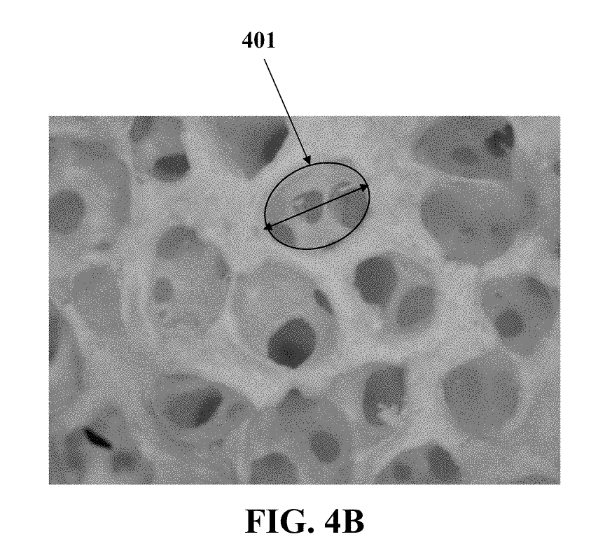

FIGS. 4A and 4B shows the microscopic images of macrostructure of an example of the in-situ fabricated porous scaffold which were taken by using an optical microscope with a magnification of 47 (FIG. 4A) and 200 (FIG. 4B). The specified pore 401 in FIG. 4B has a diameter with a size of about 691 micrometers.

In order to evaluate pore interconnectivity of the fabricated scaffold, the porous scaffold which made up of PLGA was soaked in colored pigment suspension for staining the interconnected pores; and then it was subjected to centrifugation for penetration of the pigments into the pores. After removal of excess pigments, the scaffold was sectioned; and all the sections were dried by using a sterilized dry cotton gauze. After that, images of each section of the stained scaffold were taken by using a stereo optical microscope.

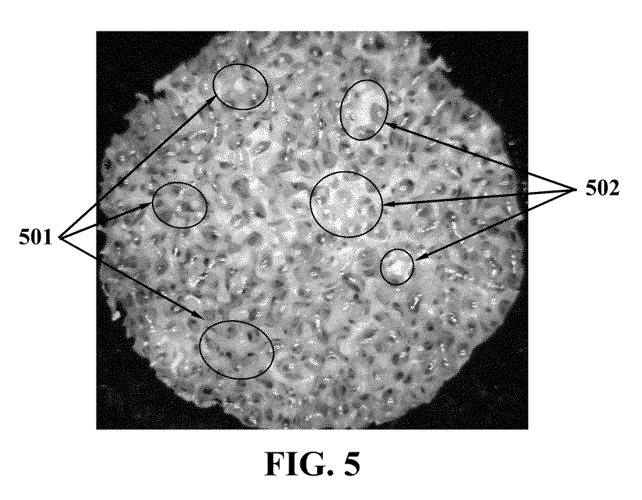

FIG. 5 shows the images of the scaffolds after the interconnectivity assay. This image shows that the scaffold was composed of pores with a proper interconnectivity with dark color. Colored areas 501 indicate the interconnectivity of pores, which means the pores in these areas have permitted the penetration of blue pigments during the centrifugal force either directly or via neighboring pores, while uncolored areas 502 which shown in bright white color indicate the lack of pigment penetration into their pores and low pore interconnectivity in these areas.

Referring to FIG. 5, it seems that the produced scaffold has about 90% interconnectivity. Furthermore, given that the PLGA is a biodegradable polymer and it degrades over time, the pore interconnectivity of the scaffold increases during degradation of PLGA in patients' body.

In order to perform the mechanical analysis of the porous scaffolds, six specimens of the in-situ fabricated PLGA scaffolds were prepared in a cylinder-shaped mold with a diameter of about 6 mm and a height of about 5 mm, pursuant to the teachings of the present disclosure. Mechanical assay was performed on the specimens by using a scanning tunneling microscopy (STM) at three times (about 24, 48 and 72 hours after scaffold fabrication). A scanning tunneling microscope was used to compress the PLGA scaffolds at a constant rate of about 1 mm/min.

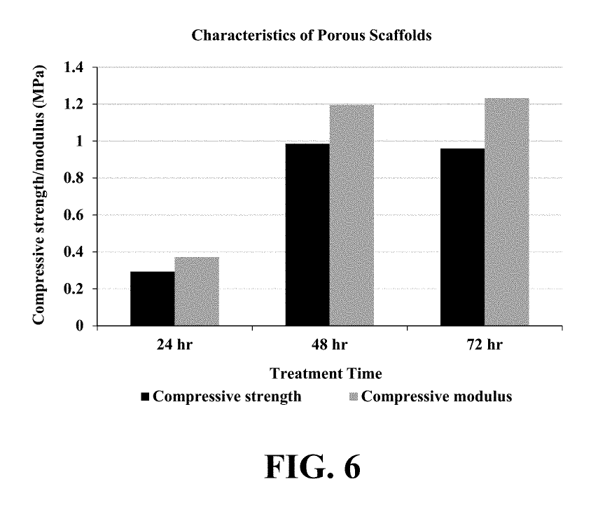

After that, stress versus strain curves were generated for each scaffold and then, the compressive modulus of the specimens were calculated according to the slope of the linear region of stress versus strain curves. FIG. 6 shows a diagram of compressive strength and compressive modulus of the porous scaffolds at three time points after their fabrication. Referring to FIG. 6, the compressive strength increased from about 0.3 MPa (at about 24 hours) to 0.985 MPa (at about 48 hours); also compressive modulus ranged from about 0.4 MPa to about 1.2 MPa (p<0.05). No significant difference was observed between the compressive strength and the compressive modulus of the specimens treated about 48 and about 72 hours after fabrication (P>0.05).

What is claimed is:

1. A method comprising:
mixing a polymer, an organic solvent and a porogen such that an initial paste is formed;
in-situ shaping the initial paste such that a shaped paste is formed;
creating a plurality of channels within the shaped paste and removing the organic solvent from the shaped paste such that a solidified perforated paste is formed; and
leaching out the porogen from the solidified perforated paste such that a porous scaffold is formed.

2. The method of claim 1, wherein, creating the plurality of channels within the shaped paste and removing the organic solvent are performed substantially simultaneously.

3. The method of claim 1, wherein removing the organic solvent is performed after creating the plurality of channels.

4. The method of claim 1, wherein the porous scaffold has a pore size between about 50 micrometers and about 1000 micrometers.

5. The method of claim 1, wherein the porous scaffold has a uniform porosity.

6. The method of claim 1, wherein the porous scaffold has high pore interconnectivity.

7. The method of claim 1, wherein the polymer includes a biodegradable and biocompatible polymer.

8. The method of claim 7, wherein the polymer is selected from the group consisting of polylactide (PLA), poly(lactide-co-glycolide) (PLGA), polycaprolactone (PLC), polyglycolide (PGA), polyanhydrides, polydioxanone, polyorthoester, polyurethane, polyacrylate, polycarbonate, polysiloxane, polyolefin, polyamide, polyimide, poly alpha hydroxyl acids, and combinations thereof.

9. The method of claim 1, wherein the molecular weight of the polymer is between about 2000 and about 50000 Daltons.

10. The method of claim 1, wherein the porogen is selected from the group consisting of sodium chloride, potassium chloride, potassium bromide, calcium chloride, magnesium chloride, polyethylene glycol, polypropylene glycol, sorbitol, sucrose, poly-carbohydrate, gelatin, sugar, mannitol, poly vinyl alcohol, and combinations thereof.

11. The method of claim 1, wherein the porogen has a particle size between about 100 micrometers and about 1500 micrometers.

12. The method of claim 1, wherein the porogen is soluble in water and is insoluble in the organic solvent.

13. The method of claim 1, wherein the concentration ratio (weight/weight) of the polymer to the porogen is between 1:1 (wt/wt) and 1:10 (wt/wt).

14. The method of claim 1, wherein the organic solvent is a water-miscible solvent.

15. The method of claim 1, wherein the organic solvent is selected from the group consisting of acetone, chloroform, dimethyl carbonate, N-Methyl-2-pyrrolidone, dimethyl sulfoxide, polyethylene glycol, polyethylene glycol derivatives, and mixtures thereof.

16. The method of claim 1, wherein the concentration ratio (weight/weight) of the polymer to the organic solvent is between 1:1 (wt/wt) and 1:10 (wt/wt).

17. The method of claim 1, wherein creating the plurality of channels within the shaped paste is performed using one or more needles.

18. The method of claim 17, wherein one of the needles has a needle gauge between 10 and 34.

19. The method of claim 1, wherein removing the organic solvent from the shaped paste includes a phase inversion process.

20. The method of claim 1, further comprising adding at least one active agent to the initial paste.

21. The method of claim 20, wherein the active agent includes an agent for cell induction or cell promotion.

22. The method of claim 20, wherein the active agent is selected from the group consisting of growth factors, anti-inflammatory agents, inductive agents, angiogenesis factors and tissue growth promoting agents.
